(19) United States
(12) Patent Application Publication
(10) Pub. No.: US 2020/0327564 A1
(43) Pub. Date: Oct. 15, 2020

(54) COMPUTER-IMPLEMENTED PROCESS AND SYSTEM FOR GENERATING RECOMMENDATIONS RELATING TO USER EXPERIENCES OF ENTERTAINMENT PRODUCTIONS

(71) Applicant: RE-AK Technologies Inc., Saint-Jean-Sur-Richelieu (CA)

(72) Inventors: Frédéric Simard, Terrebonne (CA); Patrick Marcotrigiano, Saint-Jean-Sur-Richelieu (CA); Somayeh Haji Kazem Nili, Montreal (CA)

(21) Appl. No.: 16/844,512

(22) Filed: Apr. 9, 2020

Related U.S. Application Data
(60) Provisional application No. 62/832,406, filed on Apr. 11, 2019.

(51) Int. Cl. G06Q 30/02; G16H 40/63

(52) U.S. Cl. CPC G06Q 30/0203; G16H 40/63

(57) ABSTRACT

A computer-implemented process and system are provided. The process and system provide recommendations for the creation or modification of a user experience of a candidate entertainment production, destined to a plurality of end-users or consumers, in view of user experiences associated to one or more other entertainment productions. Recommendations of changes to apply on the human-interpretable UX-metrics of the candidate entertainment production are automatically generated based on similarities or differences between the candidate's transformed UX-data and a target UX-template and/or ii) the human interpretable UX-metrics of the candidate entertainment production and of the UX-template.

### RELATED APPLICATION

The present invention claims priority on the U.S. provisional patent application No. 62/832,406, which is incorporated by reference in its entirety.

### TECHNICAL FIELD

The present invention relates to systems and methods for processing and analyzing biometric and/or non-biometric data collected from test-users or consumers, when participating or assisting in an entertainment production or event, including video games, movies, music concerts and the likes.

### BACKGROUND

Entertainment producers, including game studios, are interested in analyzing user-experience data to better understand enjoyment (appreciation) and engagement of their product by end users. They are also interested in using this data to guide the development of games or other entertainment media, productions or events, such as concerts or movies.

In the case of video games, the evaluation of user experience (also referred to as UX) is typically done by playtesting, which consists in having testers play the game while their biological responses are measured. They may also be asked to answer questionnaires, aimed at qualifying and/or quantifying various aspects of their experience. This information can then be used to derive requirements for game developers, to improve user experience for a target audience.

Acquiring, compiling, analysing, understanding and interpreting the data collected during playtesting requires trained experts. As new data acquisition methods are made available, through technological advancement, the volume of data to process increases. Consequently, it becomes extremely difficult, if not impossible, for experts to process this information in a timely and meaningful manner. Further, the complexity arising from integrating new sources of information impairs the ability of human experts to interpret the results and derive effective requirements for the development team.

### SUMMARY

While the methods and systems described herein are particularly adapted to video game development, they can be applied to other types of applications, including the creation or improvement of entertainment media or productions, such as, but not limited to: concerts, movies, live performing art, virtual reality or immersive experiences, etc.

In some implementations, the computer-implement method, system and storage medium described herein generate recommendations or indications with regard user experiences, as defined by UX-metrics, based on previously collected user-experiences for other reference entertainment productions.

The invention proposed aims at simplifying the process of deriving requirements from recorded user experiences, by automating and improving the process of interpreting the information using artificial intelligence techniques and comparative analytics, as well as automatically producing recommendations to serve as guidelines for development.

According to an aspect, a computer-implemented process for generating recommendations indicative of modifications to be made to a user experience associated to a candidate entertainment production being studied, destined to a plurality of end-users or consumers, in view of user experiences associated to one or more reference entertainment productions, is provided. The process comprises the steps of:
accessing a first dataset of human interpretable UX-metrics associated with the candidate entertainment production;
receiving, via a graphical user interface, a selection of the one or more reference entertainment productions, for comparison purposes, and accessing previously processed UX-metrics associated to the selection of the one or more reference entertainment productions, forming a second dataset of UX-metrics;
projecting, using a transformation module, the first and second datasets of UX-metrics into a subspace, where comparative analytics tools can be used, the subspace thereby comprising a first set of transformed UX-data associated with the candidate entertainment production and a second set of transformed UX-data associated with the one or more reference entertainment productions,
receiving target rules associated with the reference entertainment productions, the target rules being indicative of how close or how far the candidate entertainment production should be from each of the one or more reference entertainment productions, and in response, modifying the first and second sets of transformed UX-data using the comparative analytics tools, based on the target rules inputted, and generating a target UX-template;
projecting the first set of modified UX-data and the UX-template back into human-interpretable UX-metrics, using an inverse transformation module; and
generating recommendations indicative of changes to apply on the human-interpretable UX-metrics of the candidate entertainment production based on similarities or differences between i) the first set of transformed UX-data and the UX-template and/or ii) the human interpretable UX-metrics of the candidate entertainment production and UX-metrics corresponding to the UX-template.

A desired entertainment production objective can be, for example, to improve similarity with other successful productions, move away from unsuccessful productions or generate an exclusive user experience that differs from other productions, based on previously collected UX-data. The determination of whether a production has been successful or unsuccessful includes factors such as critical review, commercial success, conversion rate (player transitioning from game trial to becoming a regular player), drop rate (rate at which players stop playing), heuristic rules defined by the platform user (game studio, etc).

According to another aspect of the invention, a computer-implemented process for generating recommendations regarding a user experience associated to a new entertainment production to be created, destined to a plurality of end-users or consumers, in view of user experiences associated to one or more reference entertainment productions is provided. The process comprises the steps of:
accessing a first dataset of UX-metrics associated with the new entertainment production;
receiving, via a graphical user interface, a selection of the one or more reference entertainment productions, for comparison purposes, and accessing previously processed UX-metrics associated to the selection of the one or more reference entertainment productions, forming a second dataset of UX-metrics;
projecting, using a transformation module, the first and second datasets of UX-metrics into a subspace, where comparative analytics tools can be used, the subspace thereby comprising a first set of transformed UX-data associated with the new entertainment production and a second set of transformed UX-data associated with the one or more reference entertainment productions,
receiving target rules associated with the reference entertainment productions, the target rules being indicative of how close or how far the new entertainment production should be from each of one or more reference entertainment production, and in response, modifying the first and second sets of transformed UX-data using the comparative analytics tools, based on the target rules inputted, and generating a target UX-template;
projecting the first set of modified UX-data and the UX-template back into human-interpretable UX-metrics, using an inverse transformation module; and
generating recommendations of adjustments to apply on the first human-interpretable UX-metrics associated to the new entertainment production based on similarities or differences between i) the first set of transformed UX-data and the UX-template and/or ii) the human interpretable UX-metrics of the candidate entertainment production and of the UX-template.

According to another aspect, a non-transitory processor-readable storage medium is provided, for storing thereon instructions for causing a processor to perform the steps of either one of the processes described above.

According to another aspect, a computer-implemented system for providing recommendations indicative of adjustments to be made to a user experience of a candidate entertainment production, destined to a plurality of end-users or consumers, in view of user experiences associated to one or more reference entertainment productions, is provided. The system comprises a comparative and recommendation engine comprising:
a graphical user interface configured for receiving a selection of the candidate entertainment production and of the one or more reference entertainment productions, for comparison purposes, the candidate entertainment production being associated to a first dataset of UX-metrics, and the one or more reference entertainment productions being associated to previously processed UX-metrics forming a second dataset of UX-metrics;
the graphical user interface configured for receiving target rules associated with the reference entertainment productions, the target rules being indicative of how close or how far the candidate entertainment production should be from each of one or more reference entertainment production,
a transformation module for projecting the first dataset and the second datasets of UX-metrics into a subspace,
a UX transformation toolbox comprising comparative analytics tools for modifying the first and second sets of transformed UX-data based on the target rules inputted, and for generating a target UX-template;
an inverse transformation module for projecting the first set of modified UX-data and the UX-template back into human-interpretable UX-metrics;
the graphical user interface being further configured for displaying recommendations of changes to apply on the human-interpretable UX-metrics of the candidate entertainment production based on similarities or differences between i) the first set of transformed UX-data and the UX-template and/or ii) the human interpretable UX-metrics of the candidate entertainment production and of the UX-template.

### BRIEF DESCRIPTION OF THE DRAWINGS

Further features, aspects and advantages of the present disclosure will become better understood by reference to the following detailed description, appended claims, and accompanying figures, wherein like reference numbers indicate like elements throughout the several views, and wherein:

FIG. 1 is a schematic diagram which provides an overview of components of the system, according to a possible implementation.

FIG. 2 is a schematic diagram which illustrates the process and components by which biometric and non-biometric data is processed in order to obtain the emotional, cognitive and behavioral metrics, which are stored in a database, according to a possible implementation.

FIG. 3 is yet another schematic diagram illustrating components of the comparative and recommendation engine, according to a possible embodiment.

FIG. 4 shows a possible graphical user interface of a web application, which presents playback of emotional, cognitive and behavioral data and of a test-user captured video, while the entertainment production is displayed.

FIG. 5 shows a close-up view of a cluster dynamics recommendation pane view. The pane view presents timeseries of the clusters identifiers for a given recording of a compared dataset, as well as automatically generated recommendations.

FIG. 6 shows another possible graphical user interface showing a cluster analysis view of the web application. The graphical user interface displays cluster statistics, cluster transition probabilities and cluster alignment analysis, derived from transformed UX-metrics, according to the proposed process.

FIG. 7 is graph of a cluster flowchart, showing cluster identifiers for a selected entertainment production.

### DETAILED DESCRIPTION

Various terms used herein are intended to have particular meanings. Some of these terms are defined below for the purpose of clarity. The definitions given below are meant to cover all forms of the words being defined. If the definition of any term below diverges from the commonly understood and/or dictionary definition of such term, the definitions below should be considered for the present description.

Artificial Intelligence relates to mathematical, statistical, analytical and computational tools used to analyse data with the goal of establishing correlations, extracting information, and/or identifying patterns. Specifically, artificial intelligence algorithms often proceed in ways that mimic intelligence. A defining aspect of artificial intelligence algorithms is that they learn how to operate on their own, by being presented with data examples, often referred to as training data.

Artificial Intelligence, or machine learning, is generally used to perform one or many of these tasks: classification or pattern recognition, which consists in analysing a sample of data and assigning it a label, taken from a set of predefined labels, based on its similarity with data that were part of the training set; regression, which refers to the process of extracting a trend from a set of data, which can then be used to interpolate or extrapolate values that are not within the training set; clustering, which consists in forming groups and agglomerates of datapoints, based on similarity or other particularities.

Dimensionality reduction or expansion are methods by which a sample of data can be re-mapped to a reduced or expanded number of variables according to a transformation process, which has been defined analytically or using artificial intelligence. Data projected in a multidimensional space will generally have mathematical properties and characteristics that can simplify its analysis.

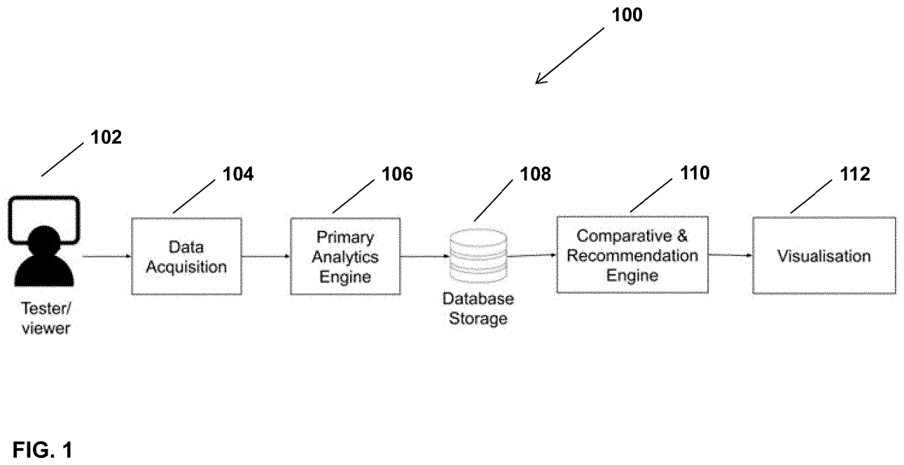

Referring to FIG. 1, a general overview of the proposed process 100 is illustrated, for providing recommendations to create, improve or modify the user experience of a candidate entertainment production, compared to one or more existing entertainment productions, for which user experience data has been collected. In the present description, the expression “entertainment production” encompasses video games, but also events in which end-users can assist or participate, including music concerts, movies, TV shows, plays, live performing arts, etc. The entertainment production can be a new production being put together, or a production under development, such as a prototype or pilot production, and is typically destined to a plurality of end-users or consumers. A video game is a software that provides an experience to the player. It can be defined by its type and attributes including, but not limited to: action; real-time strategy; turn-based strategy; simulation; adventure; puzzle; shooter; battle royale; role playing game; educational; multiplayer; and sports.

User experience relates to the interactions and reactions of users when consuming a product. For example, with regards to video games, user experience encompasses what is felt by a player while they play or interact with the video game. User experience encompasses how pleasant or frustrating, easy or difficult, entertaining or boring, surprising or unsurprising, engaging or not engaging, the interaction with the video game is, from the player standpoint. User experiences can be quantified and qualified, using biometric and non-biometric data, collected from test-users interacting or assisting to an entertainment production. The biometric and non-biometric data can be processed into UX-metrics that provide information on the cognitive, emotional and behavioral state of the users when interacting with a production (video game, play, etc.)

The recommendations and/or guidelines provided by the computer-implemented process are generated in view of past user experiences associated with one or more other entertainment productions, for which biometric and/or non-biometric data has been previously collected and processed. Recommendations and/or guidelines can include indications provided to operators, producers, game developers and/or product owners, indicating how human-interpretable metrics, but also how clusters of statistical data derived from human-interpretable metrics, must be set or adjusted to achieve a target user experience for a given entertainment production. For example, the recommendations can help generate adoption of a target audience for a given type of production event, help replicate the user experience of a blockbuster videogame, or distance the targeted user experience from the combined user experiences of a group of movies. More specifically, the recommendations provided by the proposed process and system can include indications as to how similar or dissimilar the mental state duration and/or mental state transitions of a candidate/under-development entertainment production are, when compared the mental state durations and/or transitions of selected entertainment production(s). The recommendations can also include indications or information on how close or how far UX-derived cluster identifiers from the candidate entertainment production are from UX-derived cluster identifiers of selected entertainment production(s).

Biometric data relates to biological measurements and calculations. As regards to biometrics (or biometric data) relating to user experience, they include biomarkers indicative of latent variables related to cognition; emotions; attention; stress and state of arousal; which can be experienced consciously or subconsciously by the test-users. Biometric data may include: electrodermal activity, electroencephalogram signals, near-infrared spectroscopy (NIRS), eye movements and characteristics, heart rate and heart-beat characteristics, voices signals, body movements and/or facial expressions. These signals often require further processing in order to be interpretable by human experts.

Non-biometrics measurements relating to user experience encompass, but are not limited to: keystrokes; mouse movements; contextual information; software events; time in general, and time periods of an entertainment production; environmental sound, environmental noise and/or soundscape, including character voices, music, sound effects, etc.

Thus, “biometric and non-biometric data” refers to data recorded or collected using sensors and other type of equipment (such as keyboard, mouse, cameras, etc.), represented in their raw form, with minimum amount of processing. “Processing” encompasses applying transformations and/or treating the data, using programmed routines, software modules and/or functions to derive UX-metrics, which can be represented as an indication of the cognitive, emotional and/or behavioral state of a person.

An “end-user” refers to any person likely to consume an entertainment production, while a “test-user” refers to a player, a tester, a viewer, a consumer, a participant, a spectator or any person from which biological and non-biological signals and data is collected. A “test-user” can be a person who plays a game being evaluated, or who watches a TV show pilot. Test-users can be defined by several characteristics, including age, gender, type of gamer (casual, intermediate, expert, professional). Other non-related user data can also be used in the context of the invention, such as the recording context (casual gaming; user research context; in training; in competition). “Test-users” provide biometric information through sensors and equipment detailed below, as will be explained in more detail below. A tester or viewer 102 is identified if FIG. 1, at the beginning of the process. An “operator” is a user of the system and processes detailed below. The term “operator” may also be referred to as a “researcher”, “UX developer”, “manager”, “team leader” or “developer”.

### Overview of Components of the System

Referring to FIG. 1, according to a possible implementation, the proposed system includes a data acquisition system 104, a primary analytics engine 106, a database storage 108, a comparative and recommendation engine 110, and a visualisation module 112.

The data acquisition system 104 is the system used to collect biometrics and non biometrics data from test-users. The primary analytics engine 106 is a set of algorithms used to process the raw data collected by the data acquisition system, to remove artefacts and undesired sequence of information, and extract therefrom, relevant behavioral, emotional and/or cognitive UX-metrics that can be used for further processing. The UX-metrics extracted from the UX-raw data is then store in a repository or storage database 108. The comparative and recommendation engine is a collection of software modules and tools that transforms UX-metrics from reference/selected entertainment productions, and, in most implementations, “under-study” UX-metrics, and generates recommendations for achieving a target user experience. The visualisation system or module 112, is the module that displays the recommendations, through different windowpanes where modifications or adjustments to be made on different UX-metrics and/or clusters of data derived from UX-metrics, are shown.

In the illustrated embodiment, the process integrates steps from the data acquisition to the recommendation presentation. However, in other implementations of the system, the data acquisition system 104, the primary analytics engine 106 and the database storage 108 do not necessarily form part of the system 100. What is needed is to have access to the UX-metrics data storage or other equivalent storage medium. The storage database remains, in possible implementations, owned and/or operated by a third party (company, person or entity) who stores, grows and maintains a main database 108, which includes the raw biometric and non-biometric data, and/or UX-metrics derived from the raw biometric and non-biometric data, associated to a plurality of entertainment productions, collected from a plurality of test-users.

### From Biometric and Non-Biometric Data to Emotional, Cognitive and Behavioral UX-Metrics

Data Acquisition

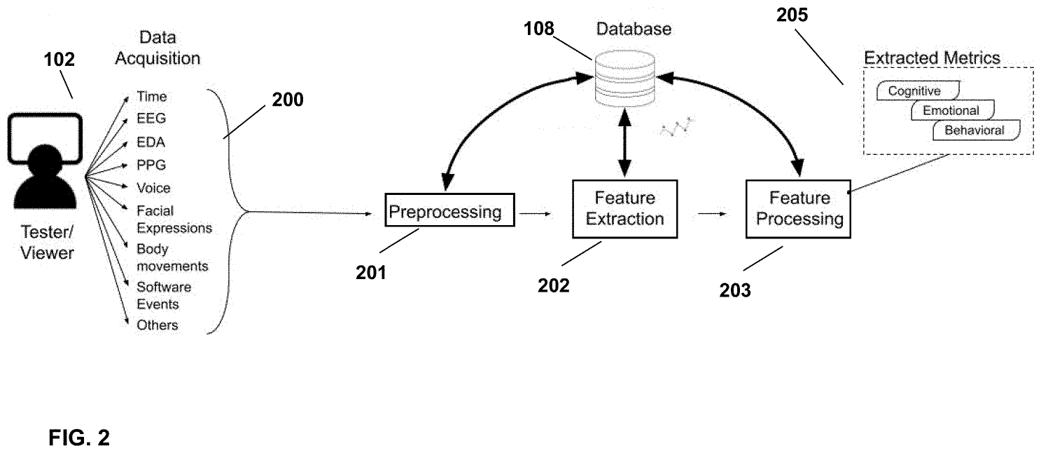

Referring now to FIG. 2, the data acquisition system can include components and sensors for recording of the biometric and non-biometric data of test-users, while she or he plays a video game or interact with other types of entertainment content. Recorded data, identified by numeral 200, can include, but is not limited to: time, electroencephalogram (EEG), electrodermal activity (EDA), photoplethysmography (PPG) or electrocardiogram (ECG), voice and ambient sounds, facial expressions, eye movements, body movements, software or video games events (such as player death, level beginning or ending, progression in tutorial stages, puzzle solving step, combat situation, in-game movement and exploration, game active scene), and others (age, gender, type of gamer, keystrokes and mouse movements). Data acquisition can be performed using an array of sensors such as: EEG headset, EDA sensor on the wrist, fingers, forehead, or foot; PPG or ECG sensors; infrared cameras; microphones; and other software tools.

Preprocessing Biometric and Non-Biometric Data

Still referring to FIG. 2, the collected biometric and nonbiometric data 200, is preprocessed, using the algorithms of a preprocessing module 201, to remove artifacts in the collected data, such as eye-blinks in EEG, movement and cardiac artifacts in EEG, EDA, PPG; line noise in EEG, EDA, and undesired information (such as unnecessary game events). Preprocessing of the collected data can be performed using time or frequency domain filters, trained machine learning or statistical tools (including for example: Independent Component Analysis (ICA) and/or neural networks). Some of the data may need to be reconstructed to fill-in missing samples or repair corrupted information. Data from various sources also require time alignment to ensure the different samples of biometric and/or non-biometric data are all synchronized.

Feature Extraction

Still referring to FIG. 2, once the UX-raw data has been preprocessed, the feature extraction step can be performed, using algorithms of the feature extraction module 202. Features are derived values that describe the biometric and/or non-biometric data after it has been processed, to extract meaningful information. Extracting features from the preprocessed data allows generating informative and non-redundant values that facilitates further analysis thereon. Some of the recorded data is already suitable for being processed by advanced tools, but for other data, it is better to extract meaningful features before proceeding with further analysis. Feature extraction operations may include: power bands calculation (applied to preprocessed EEG and/or NIRS data), heart-beat identification (applied to PPG and/or ECG data), Skin Conductance Responses (applied to EDA data), facial landmarks (applied to facial expressions), gaze location (applied to eye movements data), skeletal model (applied to body movements data), statistical analysis (applied to keyboard strokes, mouse, game events data), spectrogram (applied to voice and sound data) and text transcript (applied to voice signal/data). This step generally requires analytical tools including, but not limited to: Fourier Transform, Wavelet, statistical analysis, information theory analysis, but also trained models (such as neural networks, support vector machine, linear discriminant analysis, principal component analysis, factor analysis, covariance matrix, etc.)

Feature Processing

Still referring to FIG. 2, extracted features represent the data in a way that is suited for further processing using advanced analytical tools. Feature processing consists in extracting, from the extracted features, meaningful indexes, using a feature processing module 203. Indexes represent features that have been processed to extract interpretable, psycho-physiological metrics. Indexes can thus also be referred to as human-interpretable UX-metrics and provides insightful and interpretable information about the user experience felt by the end-user. This process can be done using analytical equations or can rely on machine learning (ML) models trained for regression or classification. UX-metrics can be provided as values on a predefined scale. For example, on a scale of 0-10, 0 would represent extreme sadness and 10, extreme happiness. In the present application, indexes (or UX-metrics) are grouped in three (3) categories 205: cognitive indexes or metrics, including one or more of: relaxation state, engagement, attention, working memory, mental workload, cognitive dissonance, etc.; emotional indexes or metrics, including one or more of: emotional valence, arousal or emotional intensity, happiness, sadness, anger, contempt, fear, disgust, surprise, heart-rate and heart-rate variability, stress and behavioral indexes or metrics, including one or more of: keyboard entropy, movements intensity, gaze heatmap, all of which are insightful and interpretable UX-metrics about the user experience as felt by the player or viewer. Of course, other types and grouping of indexes/metrics can be considered, without departing from the present invention.

Data, features and indexes are stored in the database 108, for future reference, but also to be used as the source of training datasets for preprocessing, feature extraction and feature processing statistical and machine learning models. Trained models can themselves be stored in the database.

### Comparative Analytics and Recommendation Engine Overview

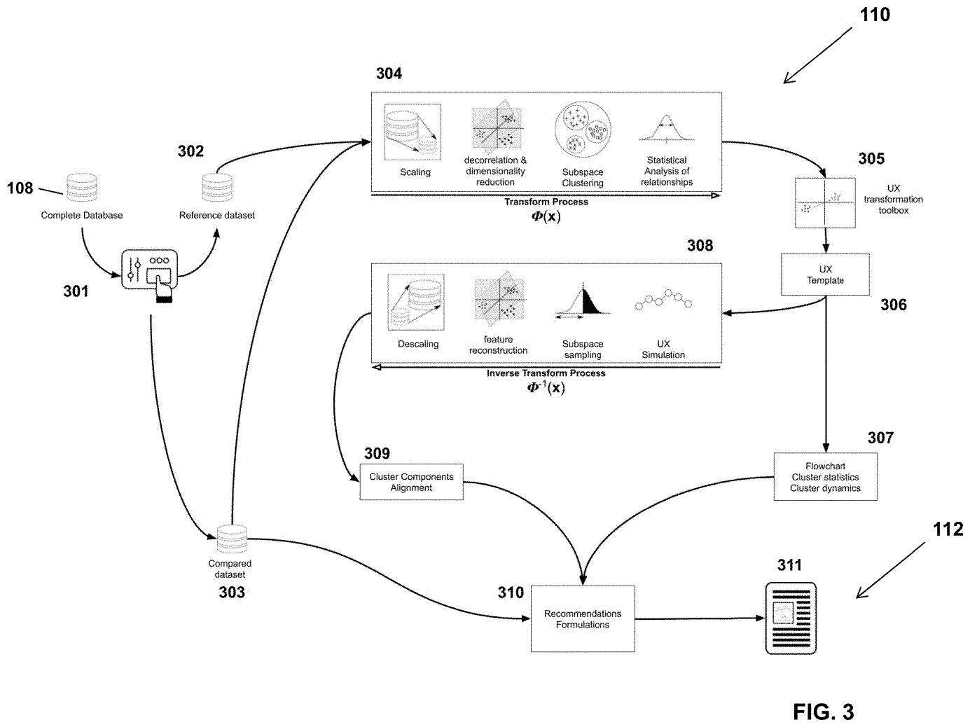

Referring now to FIG. 3, in a possible embodiment, the comparative and recommendation engine 110 is used to compare a subset of the UX-metrics (or UX-indexes) stored in the database, where the subset can be referred to as the “reference dataset”, with UX-metrics obtained from an initial or in-development version of an entertainment production, referred to as the “compared dataset”, to automatically generate UX-based recommendations to improve, change or alter the user experience of the new or in-development entertainment production. The “new” or “in-development” entertainment production can also be referred to as a “candidate” entertainment production. The UX-metrics dataset of the candidate entertainment production can be referred to as a “first dataset” and the UX-metrics of the reference entertainment productions can be referred to as a “second dataset”. The comparative and recommendation engine 110 can be used to compare UX-metrics of different versions of the same entertainment production; of different entertainment production events (for example: to compare a new video game in development to three or four videogames of the same type); but also to compare a given entertainment production to completely different entertainment productions (for example, to compare a video game to a mix of movies and music concerts.

Still referring to FIG. 3, possible components of the analytics and recommendation engine 110 comprise: the transformation module 304, the UX transformation toolbox 305, and the inverse transformation module 308. The comparative and recommendation engine 110 can be executed by one or more processing devices, such as computers and/or servers. The one or more processing devices include memory, processors and input/output ports to communicate with the database, and possibly with other devices, such as tablets and/or laptops on which the recommendations and indications can be displayed. The engine 110 and its different modules can run locally, or remotely, in a distributed manner, on cloud-based servers. The comparative and recommendation engine can be stored on a storage medium and executed as a set of instructions by one or more processing devices.

In order to compare UX-metrics associated with the entertainment production under study with previously processed UX-metrics associated with other entertainment productions, different transformation steps are performed on the UX-metrics, using machine learning models and/or statistical analysis tools. Different transformation submodules or tools, part of module 304, are used to project the UX-metrics to a subspace where it can be modified using rules and tools from the UX transformation toolbox 305. In this subspace, the UX-metrics are converted into a set of statistical values, that are not interpretable by humans, but that can be manipulated to group them and/or adjust them toward a target comprising sets of other statistical data. Once the projected UX-metrics have been modified, either automatically using Al models, or via inputs collected from an operator (game studio employee, production manager, etc.), the projected and modified UX-metrics is projected back to the human-interpretable domain of representation, using the inverse transformation module 308. Recommendations can be generated based on the projected and modified UX-metrics, with module 308, and also based on the “retroprojected” UX-metrics, using module 309. The projected and retro projected UX-metrics can be combined and visually displayed in a graphical user interface 311. The different steps of the transformation process are explained in more detail below.

As examples of possible recommendations, modifications and adjustments to be made to the cognitive, emotional and/or behavioral UX-metrics can be provided, so that the UX experience of an in-development entertainment production be closer or farther away from selected entertainment productions. If the target entertainment production is a completely new production, the template of the UX-experience, about the cognitive, emotional and/or behavioral UX-metrics can be provided, using only a group of selected entertainment productions. In order to provide recommendations for a new entertainment production for which there is no initial dataset, the UX-metrics of one existing entertainment production can be used and modified and compared to the UX-metrics of other selected entertainment productions.

Comparing user experiences in terms of cognitive, emotional, attention, state of arousal and contextual information rapidly becomes extremely overwhelming as the number of samples and characteristics to be considered increases. Existing solutions, which require human experts to conduct the analyses, reduce or limit the size and type of data that is used, which affect the resulting conclusions and increases the time taken to conduct such analysis. In addition, comparing the user experiences using biometric and non-biometric raw data, or using untransformed UX-metrics is misleading, as some of these variables may not be independent. When analyzing different types of data, serially, one after the other, rather than simultaneously, as possible with the proposed system, changes made to one type of data can impact other related data, which in turn affects the overall analysis. As a result, analyses made directly on human interpretable UX-metrics are limited, and human-made recommendations are prone to errors and misinterpretations. In summary, comparing the user experiences in terms of cognitive, emotional, attention, state of arousal and contextual information is impractical for experts, and not mathematically-relevant.

Using machine learning and artificial intelligence modules, including for example principal component analysis; independent component analysis; linear discriminant analysis; tangent-space linear discriminant analysis; factor analysis; gaussian processes factor analysis; and/or deep-learning; the data is processed to define a subspace that optimises some of mathematical attributes of the processed UX-metrics, such as: mutual information, decorrelation/correlation, statistical distribution, etc.

The proposed process and methods involve using a transformation module 304 to project the reference and compared datasets in a mathematically-relevant subspace where the transformed cognitive, emotional, attention, state of arousal and contextual UX-metrics can be processed and manipulated using analytical tools which would otherwise not be applicable to standard human-interpretable UX-metrics or unprocessed biometric data. In the subspace, the transformed datasets can be optimized for selected mathematical attributes, such as reduced dimensionality, mutual information, decorrelation/correlation, clustering, as examples only. Transforming the reference and compared UX-metrics datasets in the subspace enables using a larger array of mathematical operations and analytical tools to process the transformed datasets, such as the flowcharts analysis, the cluster statistics and the cluster dynamics, as detailed below. Once transformed, the different UX-metrics or parameters, associated to different time buffers or time series in the lifetime of the entertainment production, are converted into a set of statistical data, that can be manipulated, for example for forming clusters, and that can be modified, to bring the transformed data closer or farther away from transformed data of one or more reference productions.

Projecting the reference and compared datasets in the subspace not only simplifies processes applied thereon, but advantageously prevents experts from imposing conscious or unconscious preferences on how the compared dataset should be modified or improved. The UX transformation toolbox provides tools allowing operators to input high-level rules that will define how the compared dataset must be modified. High-level or target rules can be for example that the user experience of the candidate/in development video game matches exactly the user experience of a previous blockbuster videogame. Another example of a possible target rule can be to have the user experience of the candidate entertainment production be a mix of user experiences of different entertainment productions A, B and C. Al models can be used to determine the modifications to be made to the compared dataset to bring it closer or away from datasets of previously selected reference entertainment productions. The target rules can correspond to weights attributed to each of the selected entertainment productions. The weights can be positive, to work as attractors, such that the transformed UX-metrics be brought closer to some of the reference entertainment productions, or negative, to work as repulsors, to distance the transformed UX-metrics from other of the selected reference entertainment productions. The selected rules, applied onto the datasets, can then be transformed into practical recommendations or indications, which will form the UX-target or UX-template. The UX-target or UX-template is thus a set of target values that the transformed UX-metrics should correspond to.

In the subspace, analytical transformations can be applied to the data, relationships can be highlighted, and transformations can be recommended, but the transformed data doesn't provide cognitive, emotional and behavioral targets, since data in subspace is an abstract representation (a set of statistical data) of the human-interpretable UX-metrics. As such, the inverse transformation function is required to retro-project the UX template, determined in the subspace, back to human-interpretable target values of cognitive, emotional and behavioral UX-metrics. The inverse transformation enables generating a cluster alignment analysis, as will be explained later in the description.

Reference Database

Still referring to FIG. 3, according to a possible embodiment, large sets of biometrics and non-biometric data are collected in different environments and contexts, for different types of entertainment productions. As explained previously, the biometric and/or non-biometric data may be captured from sensors installed on multiple test-users participating in or attending entertainment productions, thereby generating human-interpretable UX-metrics associated to different entertainment productions. This data is processed to extract cognitive, emotional, attentional, stress, state of arousal, behavioral and contextual metrics (referred to as UX-metrics) using the primary analytics engine 106, described earlier with reference to FIG. 1. After being processed, the UX-metrics are stored in a “main”, “reference” or “complete” database 108. According to other possible implementations, the step of collected/gathering the data may be performed by a third party, and the UX-metrics used by the comparative and recommendation engine may have been previously gathered, processed and stored in the database 108.

Selection of the Reference Dataset

Still referring to FIG. 3, prior to using the comparative & recommendation engine 110, the reference and compared datasets are selected, either automatically or via a selection input entered by an operator in a pane or window part of a graphical user interface 301. Selection of the reference dataset can be made to consider factors such as: demography, type of testers/viewers, entertainment content recorded and other factors. In one possible embodiment, the selection is made via the graphical user interface of the application, through which an operator chose different parameters that will define the reference dataset.

The reference dataset 302 thus corresponds to a collection of UX-metrics associated to one or more entertainment productions selected for comparison with the UX-metrics of the candidate production. The selection of the reference dataset can be made by detecting, via a graphical user interface, a selection of one or more entertainment productions, from a larger collection of entertainment productions. The reference datasets associated to the selected entertainment productions can be retrieved automatically from the storage database. The compared dataset, 303, is the dataset under study and for which recommendations will be produced. For new productions, a subset of the UX-metrics, associated to a given production for example, can be used as the “compared” dataset. There are no theoretical limits to the size of each dataset, although the reference dataset is likely to be larger than the compared dataset.

In summary, a subset of previously processed UX-metrics associated to the one or more other entertainment productions is retreived from the complete/larger database 108, to form the reference dataset. The selection 301 can be made based on predetermined criteria, such as the type of player, the recording context, the target end-users, the type of video game, commercial success, etc. The selection can be made automatically by a software and/or Al module, or via the capture of parameters selected though a user interface.

Transform Process

Referring to the top portion of FIG. 3, the transformation process (or simply “transform” process) is used to create an application-specific representation space into which the reference dataset 302 and the compared dataset 303 can be projected. The transformation module 304, comprise different submodules or tools to conduct some or all of the following operations:

Data scaling is an operation made to ensure that the UX-metrics are scaled, which may be necessary for the next steps to be conducted properly. Possible scaling operations include: normalization (quantile transform), division by maximum value, standardization, and minimum-maximum scaling. At this point, the UX-metrics are transformed using a selected function F(x), but are still proper UX-metrics representations:

X_{scaled}=F(X)

Decorrelation and dimensionality reduction are operations performed to simplify the scaled metric data into informative decorrelated latent variables. The decorrelation operation is a mathematical transformation Q(x) that reduces information redundancy. Dimensionality reduction is used to reject latent variables that contain little pertinent information. These steps can be executed using supervised and/or unsupervised algorithms, such as Principal Component Analysis, Linear Discriminant Analysis or Factor analysis. At this point, the cognitive, emotional and behavioral metrics are usually no longer interpretable under a psychophysiological framework. Their representation, L, is mathematically pertinent, but abstracted from cognitive, emotional and behavioral labelling:

L=Q(X_{scaled})

Subspace clustering is performed to agglomerate or cluster the scaled, decorrelated and reduced UX-metrics into groups/clusters based on proximity, density or other metrics of similarity. Following this step, each datapoint of the recorded timeseries L(t) associated to the metrics are assigned to a cluster identifier Cᵢ. The clustering step can be performed using various clustering algorithms, including the following: k-means, DBSCAN and gaussian mixture. At this point, the transformed UX-metrics data is represented as timeseries of cluster identifiers, that can also be referred to as “mental states” C(t).

C(t)=cluster(L(t))

Statistical analysis operations can then be conducted to model various aspects of the transformed UX-metrics, now represented as a timeseries of mental states. The transformation module 304 can include statistical tools to compute the probability density function of leaving the mental states, the transition probabilities between mental states and the empirical statistical distribution of latent variables in a cluster. The transition probabilities are calculated by considering the sequence of the transitions, where the sequence transitions are modeled as a memory process, as opposed to a memoryless process. For each formed cluster, the empirical statistical distribution of each of the latent variables can be defined.

Statistical modeling can be done while preserving the relationship between the data and the context during which it was recorded (such as during a video game or a movie). The proposed system and process thus allows generating as many complete statistical models as there are entertainment productions, may it be different productions or different versions of the same production.

Following the transformation process, each entertainment production has its own statistical representation of: mental states transitions and durations and/or cluster (mental state) identifiers. The system provides a UX-transformation toolbox 305, for adjusting the statistical information (i.e. the mental states transitions and durations, cluster identifiers, etc.) to define a novel UX template, 306. The UX-target or UX-template is thus a set of target values or parameters associated to the mental state transitions and durations, and clusters. Examples of operations that can be performed using the UX transformation toolbox 305 include:
Removal of undesired mental states, through a reduction or nullification of transitional probabilities leading to the undesired state. This operation can be performed to move the compared data away from a reference data of a given entertainment experience, which exhibits these states, but which are considered as undesired mental states.
Averaging cluster statistics related to a group of desired entertainment experiences. This operation is performed to create a UX template which is a mixture of other desired recorded UX.
Augmentation of the prevalence of selected mental states, through the adjustment of transitional probabilities. This operation modifies the UX template such that mental states that are under-represented, in view of predefined objectives, are now better represented.
Creation of mental states sequences. Through the adjustment of statistical parameters, a structure of the transitional probabilities can be set, so as to insert a desired sequence of mental states.

The proposed system and process allows for 1) the creation of novel UX templates generated based on the reference data selected (with or without using compared data 303) or 2) an adjusted UX template, resulting from adjustments made, in the subspace, on an initial or compared dataset 303, based on the selected reference dataset.

The UX-transformation tools can include or use the following algorithms or tools: clustering algorithms (DBSCAN, KNN, etc.), regression tools (linear, polynomial and non-linear regression, logistic regression, gaussian processes, deep neural network, etc.) classification tools (naive Bayesian, neural network, support vector machine, forest tree, etc.), statistical tools (statistical modeling, averaging, robust statistics, etc.), and linear algebra operations (translation, rotation, scaling, etc.).

As an example only, using tools of the UX-transformation toolbox, a novel experience X′ can be defined such as : X′=0.5X+0.5A+0.5B−0.5C, where A, B and C are different video games, and X is an initial version of the video game being developed and under study. Weights associated to each production can be set by an operator when defining the target UX for the candidate entertainment production. Considering that each game is represented by the center of mass of the recorded examples and computed in the application-specific subspace, the hypothetical game experience X′ would then be a linear mixture of all the games experiences, with games X, A, B being considered as attractor (games to share similarity with), while game C being a repulsor (game to be different from).

Production of Cluster Analytics Recommendations

Still referring to right-hand side of FIG. 3, according to a possible implementation, from the UX template defined by the previous operations, a first set of recommendations, at step 307, can be presented to an operator, using one or more of the following modules:

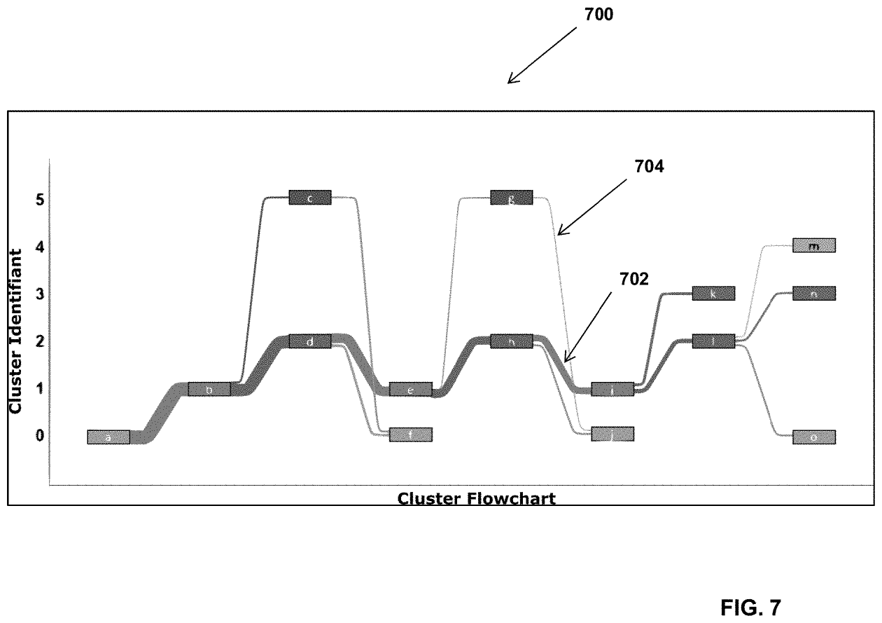

Flowchart Analysis. The flowchart analysis module provides a description of the mental states flow. This module presents how end-users (players, participants, viewers) should experience the entertainment production (game, movie, play) on average, represented in the subspace. Operators can use the flowchart analysis as a template to organize the transitions between the various phases of the entertainment production. An example of a flowchart analysis is provided by FIG. 7, where one can see the target cluster flowchart 702 of the selected entertainment production, and the current cluster flowchart 704 of candidate entertainment production, where each cluster is representative of a given mental state.

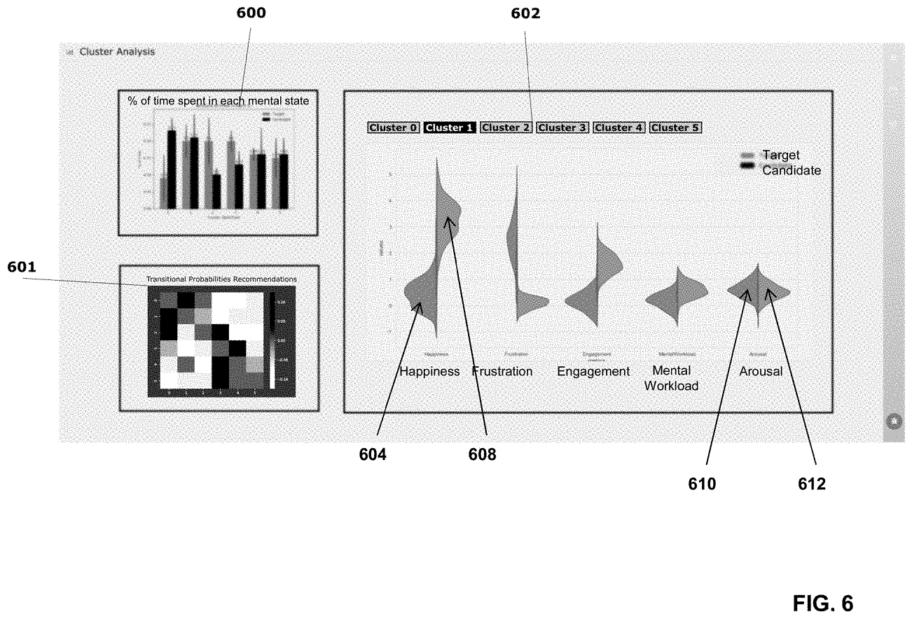

Cluster Statistics. The cluster statistics module provides a description of how much time each player or viewer spends in each mental state. This module is particularly useful to set targets on how much each mental state should be represented. Overall, this module indicates that a particular mental state is over or underrepresented, when analyzing the compared dataset vs the resulting/target UX template. Pane 600 on FIG. 6 provides a example of recommendation provided with regards to clusters, where the distribution of the different cluster identifiers from the target UX-entertainment production is represented by darker columns, and the distribution of the different cluster identifiers from the actual candidate entertainment production is represented by lighter columns. One possible recommendation could be for example to readjust the time spent in each cluster (or “mental state”) in the candidate production to match the time distribution of the clusters of target production.

Cluster Transitions Statistics. The cluster statistics module can help visualize clusters that are over or underrepresented, as shown in pane 601 of FIG. 6

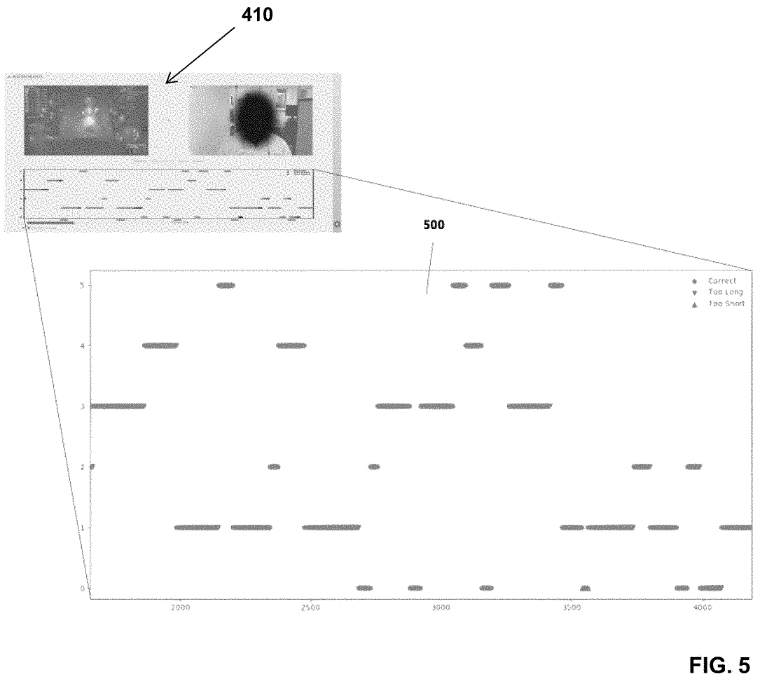

Cluster Dynamics Recommendations. This module automatically reviews the compared dataset and provides indications as to how the cluster dynamics must be modified to achieve the predefined objectives. This module or tool is compatible with the playback functionality of metrics visualization, and thus greatly helps in assessing which part of the entertainment production relates to which mental states and how the entertainment content should be changed to better match the UX template, as illustrated by pane 500 of FIG. 5.

Inverse Transform Process

Referring back to FIG. 3, once the compared dataset has been transformed using the UX transformation tools, and that the UX template generated is determined as satisfactory, (i.e that the statistical distribution of the transformed compared data as been modified in accordance to the high level rules set of the transformed reference data), the data in the subspace can be transformed back into human-interpretable UX-metrics. The following operations can be performed as part of the “inverse transformation module”, identified by numeral 308:

Mental states statistics are fed to the UX simulation engine, which generates a timeseries of cluster identifiers from the statistical parameters C′(t);
For each point of the timeseries, latent variables are sampled, using the statistical definition of the clusters, according to the cluster identifier:

L′(t)=cluster⁻¹(C′(t))
From the simulated latent variables, the scaled simulated data is recovered, using the inverse transform of the decorrelation operation:

X′_{scaled}=Q⁻¹(L′)
From the simulated scaled data, the unscaled simulated data is recovered, using the inverse transform of the scaling function:

X′_{unscaled}=F⁻¹(X′_{scaled})

Following this step, the UX template X′ is now represented in the original emotional, cognitive and behavioral space.

### Cluster Components Alignment

Still referring to FIG. 3, the Cluster Components Alignment module 309 is a tool that formulates recommendations for the compared dataset, in view of the emotional, cognitive, and behavioral target UX-metrics values. The previous recommendations (307) addressed the statistics and dynamics of the clusters, but the Cluster Components Alignment recommendation tool 309 automatically generates recommendations on how the clusters should be defined with regard to emotional, cognitive and behavioral UX-metrics values. From the recommendations generated, target UX-metrics values for emotional, cognitive and behavioral metrics can be identified, to ensure that the mental states felt during the entertainment production correspond to those of the UX template.

With reference to FIG. 6, pane 602 provides an example of recommendations made with regard to cluster components alignment. Regarding the UX-metric “happiness”, the left side of the curve 604 represents the target UX-metric and the right side of the curve 608 represents the candidate UX-metric. The curves 604 and 608 are offset and different from one another, meaning that the happiness UX-metric for the candidate production is not in line with what it should be to match the happiness UX-metric target. However, with regard to the “arousal” UX-metric, both sides 610 and 612 of the target and candidate curves are substantially symmetrical, meaning that the “arousal” UX-metric is on target.

Recommendation Formulation and Visualization

The recommendations 310 provided by modules 307 and 309 can be grouped (or agglomerated) and integrated in a dashboard 311.

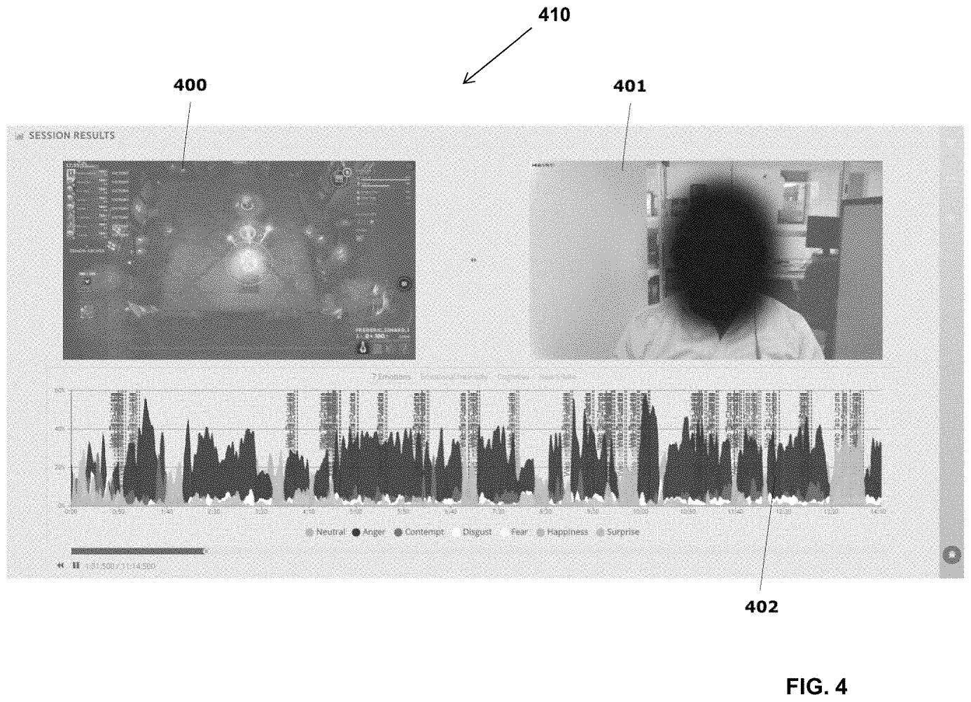

Referring to FIG. 4, the system can provide a visualization interface 410, available through a web application, which provides playback functionalities. For example, an operator can:
Play back portions of the entertainment production content (such as a sequence in a video game), as represented in the entertainment production playback pane 400;
Play back the tester or viewer's reactions, as represented in the playback reaction pane 401; and
Review and play back the cognitive, emotional and behavioral metrics, as illustrated in the metric pane 402.

Once the comparative & recommendation module has been executed, the same playback functions can be used to visualize and monitor the application of the recommendations on the cluster dynamics, as illustrated in FIG. 5, which shows the cluster dynamics recommendation view or pane 500.

Through the application dashboard (ref. 311 on FIG. 3), it is also possible to access and visualize different characteristics of the clusters, as illustrated in FIG. 6, wherein pane 600 displays cluster statistics; pane 601 displays cluster transition statistics; and pane 602 displays cluster alignment recommendations. The cluster flowchart generated by module 307 can also be displayed and visualized in the form of a detailed digital report, as illustrated in FIG. 7, in the cluster flowchart interface, 700, in a possible implementation.

Use Case Example

The following paragraphs provide an exemplary application of the process and system described above. A game studio is interested in developing a new video game, of the “Battle Royale” type. In order to define the development objectives or target for the new video game, development guidelines can be defined, using the process and system described above, based on reference data collected for the three most popular “Battle Royale” type games on the market.

A large collection of user experience data has been previously collected on a plurality of test-users, using various sensors, for different types of video games. The user experience data has then been processed by the primary analytics engine (104 in FIG. 1) to extract cognitive, emotional, attention, state of arousal and/or contextual UX-metrics. The UX-metrics are stored and maintained in a data storage (such as data storage 108 in FIG. 1).

A reference dataset is determined by selecting, from the data storage, UX-metrics associated with the three most popular Battle Royale games. The selection can be made automatically, using Al models for examples, or via a graphical user interface from which an operator's input is read, using parameters such as: type of game or type of player, in order to include only relevant reference comparatives.

If a first playable version of the video game is available, biometric data can be collected from test-users interacting with this initial version of the new game. Once processed, the UX-metrics associated with the in-development video game will form the compared dataset.

The reference and compared datasets are then processed through the transformation module, which performs the following operations:
UX-metrics from the reference and compared datasets are transformed into standard normal distribution, using quantile transformation;
the resulting dataset is processed with principal component analysis (PCA) to decorrelate the metrics and reduce the dimensionality of the reference and compared datasets, resulting in the latent variables representation;
the datasets are then agglomerated using the k-means clustering algorithm;
for each of the three games, and first playable version of the new game,
   timeseries of clustering identifiers are defined; and
   statistics relating to the dynamics of clustering identifiers are calculated.

Given that the target for the new videogame's UX has been predetermined to be a mix of the three selected “Battle Royale” games, the UX transformation tools are used to compute the average statistics of the parameters of all three games, thus creating the UX template (or UX target) of the new video game.

The averaged statistics of the parameters of all three games are then fed to the inverse transform module, in the following way:
the UX template parameters are used to generate a timeseries of clustering identifiers, representative of the newly generated UX experience;
the clustering identifiers are used to sample from latent variables distributions pertaining to each clustering identifier.
the latent variables definition is used to inverse the latent variable representation back into scaled cognitive, emotional and behavioral metrics;
the scaled metrics are unscaled, using the inverse transformation to recover the UX template that includes human-interpretable metrics for the new game being developed, where the UX template of is the targeted goal for new video game development.

The human-interpretable UX-metrics resulting from UX template are compared to the corresponding UX-metrics of the compared/candidate dataset. This analysis helps define how far the current version of the game being developed is from the targeted experience and how it should be adapted to meet the objectives.

During the development of the new video game, UX-metrics can be processed for each iteration of the game and used for comparison against the target UX-template to determine how close the new game matches the targeted user experience. Multiple versions of the game can be tested in order to select the one that approximates best the targeted experience.

Instead of modifying a recorded user experience, the comparative analytics engine can also be used to generate an entirely new experience, based on pre-recorded ones. To achieve this, a set of pre-recorded user experience is selected from the main database. These recordings are used to define a transform and inverse transform. Now, instead of modifying a newly recorded experience, a new virtual experience is generated from the pre-recorded experiences, using the transformation toolbox. This new experience is defined as a mixture of pre-recorded experience and can then be modified using the tools defined in the toolbox.

Once the virtual experience is defined, the inverse transformation is used to project it to a human interpretable representation, which can be interpreted by experts and used to define the requirements for a game to be developed.

As can be appreciated, comparing the user experiences in terms of cognitive, emotional, attention, state of arousal and contextual information can rapidly become extremely complex as the number of samples and characteristics to be considered increases. In addition, comparing the user experiences in terms of cognitive, emotional, attention, state of arousal and contextual information can be misleading as some of these variables may not be independent. In addition, performing operations such as clustering, regression, linear and non-linear transformation, classification on cognitive, emotional, attention, state of arousal and contextual information is at risk of encountering mathematical limitations, because these elements are not bounded by mathematical considerations.

In summary, comparing user experiences in terms of cognitive, emotional, attention, state of arousal and contextual information is far from being accurate and/or relevant.

The proposed system and method address these limitations and drawbacks by creating a subspace where the data can be manipulated, modified and processed data with analytical tools. The benefits of defining a transformation such as [symbol] (x) is that:
the UX-metrics are projected in a subspace that is mathematically pertinent (it optimizes/maximizes mathematical attributes such as reduced dimensionality, mutual information, decorrelation/correlation);
the subspace facilitates and enables a larger array of mathematical operations;
the transformed UX-metrics from different types of entertainment productions can be compared, regardless of their type (the user experience of a videogame can be compared to user experiences of a movie or even to a mix of user experiences of different movies and concerts); and
the data retroprojected from the subspace is interpretable by the users/humans and can be used to guide the development of the video game.

Although preferred embodiments of the present invention have been described in detail herein and illustrated in the accompanying drawings, the invention is not limited to these precise embodiments and numerous modifications can be made in the steps and components described, without departing for the invention.

1. A computer-implemented process for generating recommendations indicative of modifications to be made to a user experience associated to a candidate entertainment production being studied, destined to a plurality of end-users or consumers, in view of user experiences associated to one or more reference entertainment productions, the process comprising the steps of:
accessing a first dataset of human interpretable UX-metrics associated with the candidate entertainment production;
receiving, via a graphical user interface, a selection of the one or more reference entertainment productions, for comparison purposes, and accessing previously processed UX-metrics associated to the selection of the one or more reference entertainment productions, forming a second dataset of UX-metrics;
projecting, using a transformation module, the first and second datasets of UX-metrics into a subspace, where comparative analytics tools can be used, the subspace thereby comprising a first set of transformed UX-data associated with the candidate entertainment production and a second set of transformed UX-data associated with the one or more reference entertainment productions,
receiving target rules associated with the reference entertainment productions, the target rules being indicative of how close or how far the candidate entertainment production should be from each of the one or more reference entertainment productions, and in response, modifying the first and second sets of transformed UX-data using the comparative analytics tools, based on the target rules inputted, and generating a target UX-template;
projecting the first set of modified UX-data and the UX-template back into human-interpretable UX-metrics, using an inverse transformation module; and
generating recommendations indicative of changes to apply on the human-interpretable UX-metrics of the candidate entertainment production based on similarities or differences between i) the first set of transformed UX-data and the UX-template and/or ii) the human interpretable UX-metrics of the candidate entertainment production and UX-metrics corresponding to the UX-template.

2. The computer-implemented process according to claim 1, comprising collecting and processing biometric and non-biometric data captured from sensors installed on multiple test-users participating in or attending the candidate entertainment production, to generate the first and second datasets of UX-metrics.

3. The computer-implemented process according to claim 2, wherein collecting the biometric data comprises using a plurality of biometric sensors, each installed on the respective test-users and detecting biological reactions, during at least a portion of the candidate entertainment production being studied, and storing the biometric data in a database.

4. The computer-implemented process according claim 2, wherein the biometric data comprises at least one of: electrodermal activity (EDA), electroencephalogram signals (EEG); near infrared spectroscopy (NIRS), photoplethysmography (PPG); electrocardiogram signals (ECG); eye movements and characteristics; heart-rate and heart-beat characteristics; voice; body movements; reaction time and accuracy; and facial expressions.

5. The computer-implemented process according to claim 2, wherein the non-biometric data comprises at least one of: keystrokes; mouse movements; contextual information; date and time; software events; and audio environment information.

6. The computer-implemented process according to claim 2, wherein processing the biometric and nonbiometric data comprises removing artefacts and/or undesired information therefrom, extracting and processing features therefrom and generating at least one of cognitive, emotional and behavioral UX-metrics.

7. The computer-implemented process according to claim 1, wherein the human-interpretable UX-metrics of the first and second datasets correspond to values on scale indicative of at least one of: a relaxation state; an attention state; an engagement level; a mental workload; a cognitive dissonance; an emotional valence; an arousal or emotional state; happiness; sadness; anger; contempt; fear; disgust; surprise; a heart rate; a heart-rate variability; keyboard entropy; movements intensity; and gaze location and heatmap.

8. The computer-implemented process according to claim 1, wherein modifying the transformed UX-data derived from the first and second datasets comprises bringing the transformed UX-data of the first dataset closer or further away relative to the second dataset by applying at least one of the following transformations thereon: principal component analysis; independent component analysis; linear discriminant analysis; tangent-space linear discriminant analysis; factor analysis; gaussian processes factor analysis; autoencoder; and deep-learning.

9. The computer-implemented process according to claim 1, wherein projecting the first and second datasets in the subspace comprises scaling, decorrelating and reducing the dimension of the UX-metrics of the first and second datasets.

10. The computer-implemented process according to claim 9, wherein projecting the first and second datasets in the subspace further comprises clustering the scaled, decorrelated and reduced data derived from the first and second datasets.

11. The computer-implemented process according to claim 10, wherein the UX-template comprises at least one of: a set of mental states, mental state transitions and/or mental state durations, each mental state being associated to a cluster determined from the clustering of the scaled, decorrelated and reduced data.

12. The computer-implemented process according to claim 11, wherein the UX-template is generated by performing one or more of the following operations:
removal of undesired mental states, through a reduction or nullification of transitional probabilities leading to the undesired mental state;
averaging cluster statistics related to the one or more refence entertainment productions;
increasing a prevalence of some of the mental states, through adjustment of transitional probabilities; and
creation of mental states sequences.

13. The computer-implemented-process according to claim 11, wherein the step of projecting the clustered data derived from the first dataset and from the generated UX-template back into modified human-interpretable UX-metrics comprises transforming said clustered data back into at least one of cognitive, emotional and behavioral UX-metrics.

14. The computer-implemented-process according to claim 11, wherein generating recommendations of changes to apply based on similarities or differences between the first set of transformed UX-data and the UX-template comprises displaying, in a graphical user interface, at least one of: a flowchart of clusters as a function of time, cluster statistics and cluster dynamics.

15. The computer-implemented-process according to claim 11, wherein generating recommendations of changes to apply based on similarities or differences between the human interpretable UX-metrics of the candidate entertainment production and of the UX-template comprises displaying, in a graphical user interface, cluster components alignment.

16. A computer-implemented process for generating recommendations regarding a user experience associated to a new entertainment production to be created, destined to a plurality of end-users or consumers, in view of user experiences associated to one or more reference entertainment productions, the process comprising the steps of:
accessing a first dataset of UX-metrics associated with the new entertainment production;
receiving, via a graphical user interface, a selection of the one or more reference entertainment productions, for comparison purposes, and accessing previously processed UX-metrics associated to the selection of the one or more reference entertainment productions, forming a second dataset of UX-metrics;
projecting, using a transformation module, the first and second datasets of UX-metrics into a subspace, where comparative analytics tools can be used, the subspace thereby comprising a first set of transformed UX-data associated with the new entertainment production and a second set of transformed UX-data associated with the one or more reference entertainment productions,
receiving target rules associated with the reference entertainment productions, the target rules being indicative of how close or how far the new entertainment production should be from each of one or more reference entertainment production, and in response, modifying the first and second sets of transformed UX-data using the comparative analytics tools, based on the target rules inputted, and generating a target UX-template;
projecting the first set of modified UX-data and the UX-template back into human-interpretable UX-metrics, using an inverse transformation module; and
generating recommendations of adjustments to apply on the first human-interpretable UX-metrics associated to the new entertainment production based on similarities or differences between i) the first set of transformed UX-data and the UX-template and/or ii) the human interpretable UX-metrics of the candidate entertainment production and of the UX-template.

17. A computer-implemented system for providing recommendations indicative of adjustments to be made to a user experience of a candidate entertainment production, destined to a plurality of end-users or consumers, in view of user experiences associated to one or more reference entertainment productions, the system comprising:
a comparative and recommendation engine comprising:
a graphical user interface configured for receiving a selection of the candidate entertainment production and of the one or more reference entertainment productions, for comparison purposes, the candidate entertainment production being associated to a first dataset of UX-metrics, and the one or more reference entertainment productions being associated to previously processed UX-metrics forming a second dataset of UX-metrics;
the graphical user interface configured for receiving target rules associated with the reference entertainment productions, the target rules being indicative of how close or how far the candidate entertainment production should be from each of one or more reference entertainment production,
a transformation module for projecting the first dataset and the second datasets of UX-metrics into a subspace,
a UX transformation toolbox comprising comparative analytics tools for modifying the first and second sets of transformed UX-data based on the target rules inputted, and for generating a target UX-template;
an inverse transformation module for projecting the first set of modified UX-data and the UX-template back into human-interpretable UX-metrics;
the graphical user interface being further configured for displaying recommendations of changes to apply on the human-interpretable UX-metrics of the candidate entertainment production based on similarities or differences between i) the first set of transformed UX-data and the UX-template and/or ii) the human interpretable UX-metrics of the candidate entertainment production and of the UX-template.

18. The computer-implemented system according to claim 17, further comprising:
a database for storing the first and second datasets of UX-metrics associated with the candidate and the reference entertainment productions; and UX-metrics of different types of entertainment productions that can be selected as reference entertainment productions.

19. The computer-implemented system according to claim 18, further comprising:
a data acquisition system for collecting biometric and nonbiometric data from multiple test-users, from which the UX-metrics of different types of entertainment productions are extracted.

20. The computer-implemented system according to claim 19, further comprising:
a primary analytics engine for processing the biometric and non-biometric data captured into the UX-metrics associated with the different types of entertainment production.
